(19) United States
(12) Patent Application Publication
(10) Pub. No.: US 2009/0326535 A1
(43) Pub. Date: Dec. 31, 2009

(54) CUTTING INSTRUMENTS FOR ULTRASONIC BONE SURGERY

(76) Inventor: Cornelio Blus, Montemagno (Asti) (IT)

Correspondence Address: Steinfl & Bruno, 301 N Lake Ave Ste 810, Pasadena, CA, 91101, US

(21) Appl. No.: 12/375,151

(22) PCT Filed: Jul. 27, 2007
(86) PCT No.: PCT/EP07/57761

§ 371 Date: Jan. 26, 2009

(30) Foreign Application Priority Data
Jul. 27, 2006 (IT) TO2006U000113

(51) Int. Cl. A61B 17/00; A61B 17/58

(52) U.S. Cl. 606/80; 606/86 R

(57) ABSTRACT

A surgical instrument (10) serves to cut bone by vibrating at ultrasonic frequencies ranging between 22 kHz and 36 kHz. The instrument includes a titanium alloy body with a straight tang (11), a straight distal length (12) having a sharp free end (13), and an intermediate length (14) between the tang (11) and the distal length (12). The intermediate length has three successive curved portions (14a, 14b, 14c) with concavities facing towards alternate directions which are alternately opposite and perpendicular to a central axis (x) of the instrument. The intermediate (14) and distal (12) lengths are progressively flattened and get thinner from the tang (11) towards the end (13).

The present invention relates to cutting instruments for use in ultrasonic bone surgery. The instruments according to the invention are intended for use, particularly but not exclusively, in the fields of orthopaedic surgery, neurosurgery, otorhinolaringologic surgery, maxillofacial surgery, dental surgery and veterinary surgery.

The ultrasonic technique is widespread in bone surgery, where it is generally used also because it does not cause undesired cuts in the flesh. There are known surgical devices containing electrically supplied piezoelectric transducers which cause vibration of a metal instrument that can cut the bone or separate biological tissues. See, for example, patent publications U.S. Pat. No. 2,984,241, U.S. Pat. No. 4,188,952 and EP-0 238 667.

It is an object of the invention to provide a cutting instrument capable of reaching more easily inner zones of the human body which are difficult to access, such as narrow and deep recesses. Another object of the invention is to provide a cutting instrument allowing the surgeon to have a clear vision of both the cutting end of the instrument and the whole operating zone.

These objects are achieved, in accordance with the present invention, by a surgical instrument having the features defined in claim 1. Preferred embodiments of the invention are defined in the dependent claims.

The advantages of the invention will become apparent from the following detailed specification, given by way of example, reference being made to the accompanying drawings, in which:

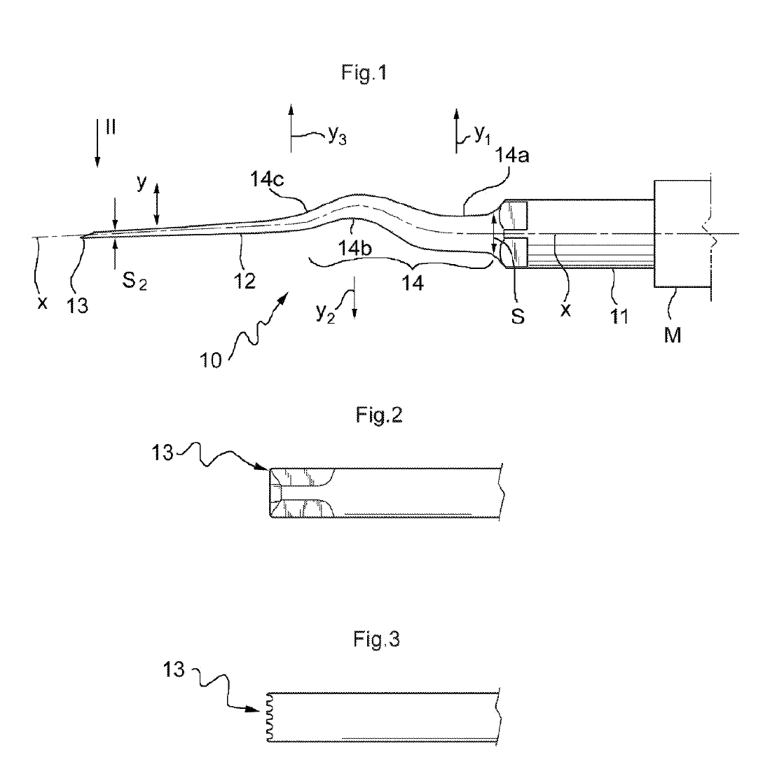

FIG. 1 is a side view of a first embodiment of a surgical cutting instrument according to the present invention;

FIG. 2 is a partial view of the free end of the instrument of FIG. 1 looking in the direction of arrow II in FIG. 1;

FIG. 3 is a partial view similar to that of FIG. 2, of a second embodiment of an instrument according to the invention;

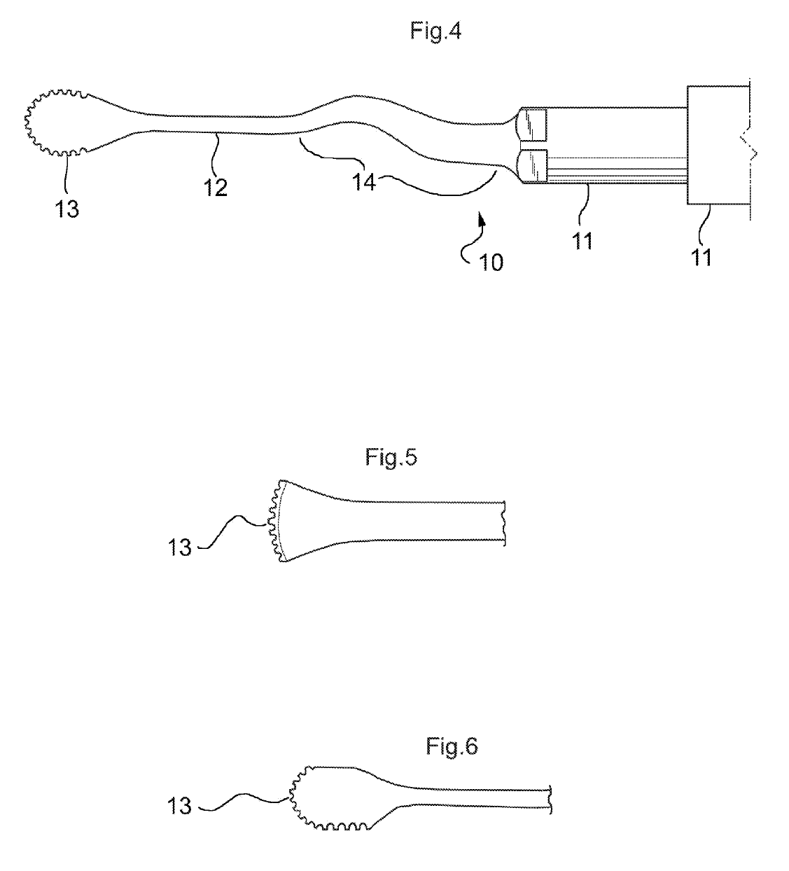

FIG. 4 is a side view of a third embodiment of a surgical instrument according to the invention;

FIGS. 5 and 6 are partial views similar to those of FIGS. 2 and 3, of a fourth and a fifth embodiment of the surgical instrument;

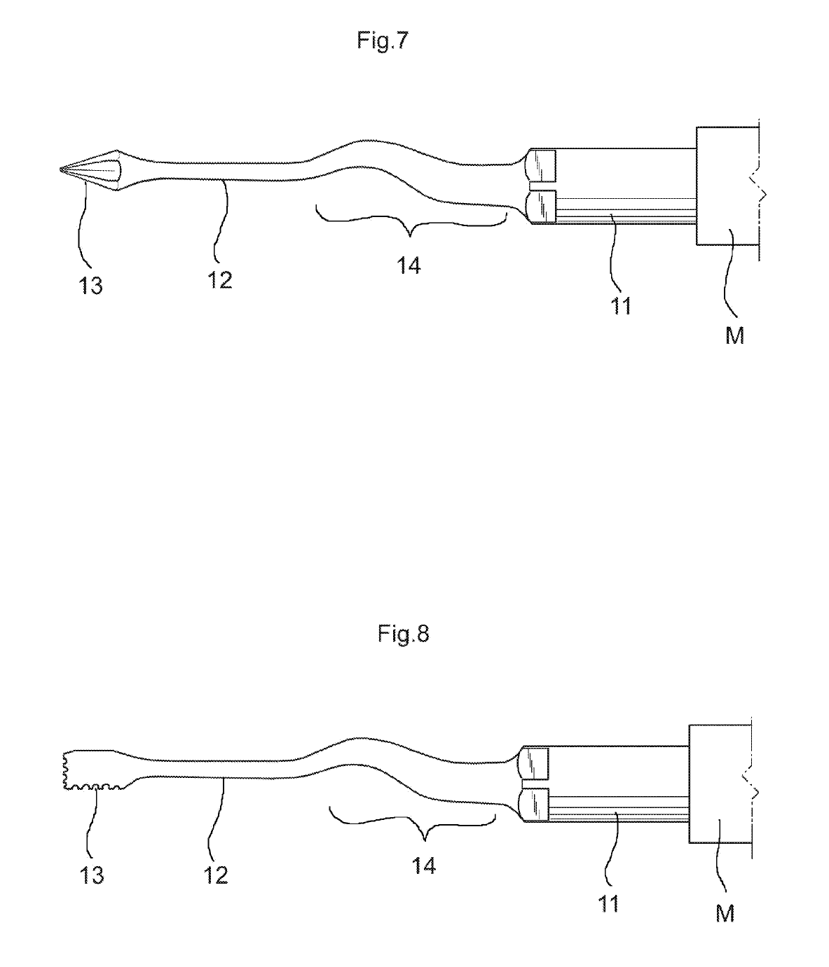

FIGS. 7 and 8 are side views of a sixth and a seventh embodiment of the surgical instrument according to the invention;

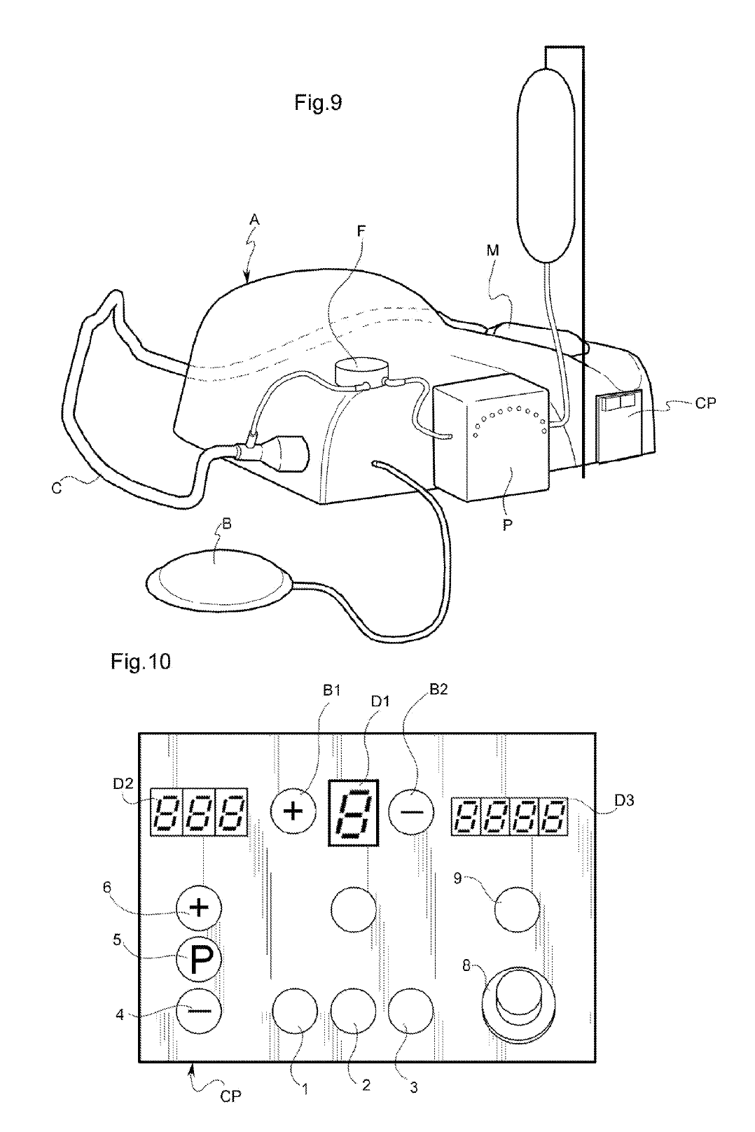

FIG. 9 is a perspective view schematically showing an apparatus for vibrating the instruments of FIGS. 1 to 9 at ultrasonic frequencies, and

FIG. 10 schematically shows a control panel of the apparatus of FIG. 9.

A first embodiment of a surgical instrument for ultrasonic bone surgery according to the invention, shown in FIGS. 1 and 2, is indicated as a whole at 10. The instrument 10, consisting of an elongate body made of titanium alloy, preferably titanium grade 5 alloy, extends along a central axis x having a succession of straight and curved lengths lying in a plane herein defined “vertical”. The instrument 10 comprises a proximal length or locking tang 11 of rectilinear shape, a thin, straight distal length 12 with a sharp cutting end 13 and an intermediate length 14 which includes three successive curved portions with respective alternating concavities, as described in detail hereinafter.

The tang 11, substantially cylindrical and having an increased diameter with respect to that of the distal length 12, is adapted to be clamped or screwed or otherwise steadily mounted onto a cylindrical handpiece M (shown only in part) that the surgeon will grip. The handpiece is part of a medical ultrasonic apparatus which electrically supplies a set of piezoelectric transducers housed in the handpiece, so as to vibrate the instrument 10 at a frequency in the ultrasonic range. The constructional and functional features of the aforementioned apparatus are not per se relevant for the understanding of the invention and will not therefore be described in detail. Suffice it here to say that said apparatus allows to adjust the input power and the frequency of the vibrations imparted to the instrument.

FIG. 9 shows by way of example an ultrasonic apparatus A with a handpiece M connected through a cable C. Indicated F is a flowmeter for measuring the flow rate of a cooling physiological solution supplied by means of a peristaltic pump P. Apparatus A is equipped with an on/off foot pedal B and a control panel CP (FIG. 10) comprised of keys 1, 2, 3 for selecting frequency channels as a function of the type of instrument being used, keys 4, 5, 6 for adjusting the flow rate supplied by the pump P, a knob 8 for selecting the frequency of the instrument mounted on the handpiece. By acting on the knob 8, one varies the frequency of the electric power supply, with a consequent impedance variation that causes a change in the frequency of vibration of the instrument. Keys B1 and B2 allow to select the power level supplied to the handpiece M, whereas displays D1 and D2 show the selected power and flow rate levels. A further display D3 shows the time the apparatus has been used. It can be reset by a key 9.

Vibrations are transmitted to the tang 11 in form of pulses along the axis x and are propagated and amplified through the intermediate length 14 and transmitted to the distal length 12 and the cutting end 13 that is brought in contact with the biological tissue to cut.

The distal length 12 extends in a direction substantially parallel to or coincident with that of the tang 11. As shown, while the tang 11 has a substantially uniform thickness, the intermediate and distal lengths 14 and 12 are progressively flattened and get thinner when measured in directions y1, y2, y3 which are perpendicular, in each point, to the central axis x and lie in the aforesaid vertical plane.

The intermediate length 14 has a first curved portion 14a with its concavity directed upwardly in the vertical plane, a second curved portion 14b with a downwardly facing concavity and a third curved portion 14c with an upwardly directed concavity radiused to the distal length 12 so that the latter is substantially aligned with or parallel to the tang 11. The bending radius of the third curved portion 14c is comparable to that of the first curved portion 14a, but definitely greater than that of the second curved portion 14b.

Preferably the thickness S1, as measured in direction y at the junction between the tang 11 and the intermediate length 14, ranges between 2.0 and 3.0 mm, whereas the thickness S2 of distal length 12, near the sharp cutting end 13 is preferably ranging between 0.4 and 0.6 mm. The overall length of the instrument, without the tang, is preferably of about 30 mm.

The instruments shown in FIGS. 3 to 8 differ from those of FIGS. 1 and 2 in the particular shape of the sharp cutting end 13, which may take different shapes according to the special purpose of the instrument (for example saw-toothed, chisel-like, triangular, with a perforating tip, etc.).

Tests carried out so far by the Applicant have proven that, owing to the shape of the instrument as described herein above, the instrument allows to cut bone located in sites otherwise difficult to reach, while letting the surgeon clearly observe the position and motion of the cutting end. More particularly, instruments made of titanium alloy have displayed surprising performances if used with ultrasonic apparatus of the above mentioned type within a frequency range between about 22 kHz and about 36 kHz. Best results have been attained using on the apparatus an input power ranging between 20 and 90 W, particularly between 42 and 90 W, with a sinusoidal wave. It has been noted that the cut resulting from the use of an instrument of the invention is particularly accurate and thin.

The above results have been attained with instruments manufactured by chip-forming machining from a blank. The same instruments are then subjected to a normalizing step in order to preserve the molecular properties of the titanium alloy. It is believed that this machining and this treatment confer better elastic properties to the instrument in terms of wider oscillations of its free cutting end.

The invention is not intended to be limited to the embodiments described and illustrated herein, which should be considered as examples of a surgical cutting instrument; rather, the invention may be modified with regard to constructional and functional details, particularly concerning the shape of the sharp cutting end.

1. A surgical instrument for cutting bone by vibrating at ultrasonic frequencies, including an elongate metal body extending along a central axis, the body lying in a plane and having a succession of straight and curved lengths, wherein the surgical instrument further comprises:
a straight tang adapted to be locked to a surgical device capable of imparting to the surgical instrument vibrations at frequencies ranging between about 22 kHz and about 36 kHz,
a straight distal length substantially aligned with or parallel to the tang, the distal length being thinner than the tang and having a sharp free end, and
an intermediate length between the tang and the distal length, the intermediate length having three successive curved portions with respective concavities facing towards directions which are alternate and substantially opposite and perpendicular to the central axis, wherein the intermediate and distal lengths are progressively flattened and get thinner when measured in directions which are perpendicular, in each point, to the central axis and lie in said plane.

2. The surgical instrument according to claim 1, wherein the surgical instrument is made of titanium alloy.

3. The surgical instrument according to claim 2, wherein the surgical instrument is made of titanium grade 5 alloy.

4. The surgical instrument according to claim 1, wherein:
a thickness of the surgical instrument, measured in a direction perpendicular to the central axis at a junction between the tang and the intermediate length ranges between about 2.0 and about 3.0 mm, and
a thickness of the distal length near the sharp free end ranges between about 0.4 and about 0.6 mm.

5. The surgical instrument according to claim 1, wherein the surgical instrument, without the tang, has an overall length of about 30 mm.

6. A method of vibrating a sharp surgical instrument so as to cut bone, the method including:
providing the surgical instrument according to claim 1;
providing an apparatus electrically supplying at least one piezoelectric transducer located in a handpiece mechanically connected with the surgical instrument;
selecting on the apparatus a frequency of said electric supply so as to cause the instrument to vibrate at a frequency ranging between about 22 kHz and about 36 kHz;
selecting on the apparatus a level of electric power supplied to the handpiece ranging between about 20 Wand about 90 W; and
electrically supplying the piezoelectric transducer with the selected frequency and power through the apparatus, thereby causing the surgical instrument to vibrate at an ultrasonic frequency ranging between about 22 kHz and about 36 kHz.

7. The surgical instrument according to claim 4, wherein the surgical instrument, without the tang, has an overall length of about 30 mm.

8. A method for performing surgery on a bone, the method comprising:
providing the surgical instrument according to claim 1;
providing an apparatus electrically supplying at least one piezoelectric transducer located in a handpiece mechanically connected with the surgical instrument;
selecting on the apparatus a frequency of said electric supply so as to cause the instrument to vibrate at a frequency ranging between about 22 kHz and about 36 kHz;
selecting on the apparatus a level of electric power supplied to the handpiece ranging between about 20 W and about 90 W;
electrically supplying the piezoelectric transducer with the selected frequency and power through the apparatus, thereby causing the surgical instrument to vibrate at an ultrasonic frequency ranging between about 22 kHz and about 36 kHz; and
applying the vibrating surgical instrument to the bone.
